(19) United States
(12) Patent Application Publication
(10) Pub. No.: US 2011/0144602 A1
(43) Pub. Date: Jun. 16, 2011

(54) Absorbent Article With Shorter Rise And Tactile Training Cue

(76) Inventors: Andrew Mark Long, Appleton, WI (US); Davis-Dang Hoang Nhan, Appleton, WI (US); Sandra Kay Knight, Omro, WI (US); Christopher Peter Olson, Neenah, WI (US); Shirlee Ann Weber, Neenah, WI (US)

(21) Appl. No.: 12/635,995

(22) Filed: Dec. 11, 2009

(51) Int. Cl. A61F 13/45

(52) U.S. Cl. 604/361

(57) ABSTRACT

A pant-like absorbent article is provided including an absorbent chassis defining a waist opening and first and second leg openings, the absorbent chassis including an absorbent assembly. The absorbent chassis has a longitudinal length and the waist opening having an unstretched circumference, such that the longitudinal length is proportional to the unstretched circumference of the waist opening by a ratio less than 0.82. The absorbent article also includes a wetness indicator for alerting a wearer to a release of liquid body exudates, the wetness indicator including a physical sensation agent responsive to liquid body exudates received by the absorbent article to facilitate a physical sensation against the wearer's skin for alerting the wearer to the wearer's release of liquid body exudates.

### BACKGROUND

The present disclosure relates to absorbent articles that include a physical sensation member. More specifically, the disclosure relates to an absorbent article such as training pants that provides the wearer with a noticeable physical sensation upon urination.

Absorbent articles such as disposable diapers and training pants are useful to absorb and contain body wastes. These products have developed to the extent that urine is quickly drawn and retained away from the wearer's skin so that the wearer remains relatively dry and comfortable. Although this improved performance enhances wearer dryness and comfort, it can reduce the wearer's ability to notice or recognize when urination occurs, especially if the wearer's attention is distracted by an activity. This is not conducive to toilet training because an important step in the early stages of toilet training is the ability to recognize when urination occurs. In an attempt to enhance a child's recognition of when urination occurs, training pants have been designed with temperature change members that provide a temperature change sensation upon urination.

Unfortunately, in certain circumstances, such temperature change members might not be completely satisfactory. For example, the element providing the temperature change sensation might not be in contact with the wearer's skin, thus limiting the effectiveness of the sensation.

Thus, there is a need for an absorbent article with a physical sensation member that is capable of more effectively providing a physical sensation to the wearer.

### SUMMARY

A problem in transferring a tactile sensation to the skin as feedback to a wetting incident has been the “bucket” design of absorbent products. This “bucket” separates the tactile element from the skin, whether the tactile element is warming, cooling, tingling, etc. Tactile sensations generally transfer poorly across an air gap. Product forms of the present disclosure reduce the rise (in the machine direction (MD) length) of the product. This effectively reduces the gap between a tactile element and the wearer's skin. As a result, the absorbent product design, including, as quantified by MD to cross-direction (CD) ratio, is significantly different from other products with tactile cues.

The present inventors undertook intensive research and development efforts with respect to improving absorbent articles, particularly in providing a wetness indicator.

The present disclosure provides a pant-like absorbent article including an absorbent chassis defining a waist opening and first and second leg openings, the absorbent chassis including an absorbent assembly. The absorbent chassis has a longitudinal length and the waist opening having an unstretched circumference, such that the longitudinal length is proportional to the unstretched circumference of the waist opening by a ratio less than 0.82. The absorbent article also includes a wetness indicator for alerting a wearer to a release of liquid body exudates, the wetness indicator including a physical sensation agent responsive to liquid body exudates received by the absorbent article to facilitate a physical sensation against the wearer's skin for alerting the wearer to the wearer's release of liquid body exudates.

The present disclosure also provides an article for personal wear, the article being capable of alerting a wearer to the wearer's release of liquid body exudates. The article includes an outer cover, an absorbent assembly, a waist opening, and first and second leg openings, the article having a longitudinal length and the waist opening having an unstretched circumference. The article also includes a liner adapted for contiguous relationship with the wearer's skin due to the longitudinal length being proportional to the unstretched circumference of the waist opening by a ratio less than 0.82, and a wetness indicator disposed between the liner and the outer cover. The wetness indicator includes an absorbent body disposed for absorbing liquid body exudates, whereby the wetness indicator swells as the absorbent body absorbs liquid body exudates, and a physical sensation element including a physical sensation agent responsive to the liquid body exudates to facilitate a physical sensation against the wearer's skin, the physical sensation agent being disposed at least one of on or within the physical sensation element such that liquid body exudates absorbed by the physical sensation element of the wetness indicator are subjected to a physical sensation by the physical sensation agent at least one of prior to and upon absorption of liquid body exudates by the absorbent body.

The present disclosure also provides a method for producing an article for personal wear, the article being capable of alerting a wearer to the wearer's release of liquid body exudates. The method includes producing a first disposable absorbent article of size n including no wetness indicator and having a size n longitudinal length and a waist opening with a size n unstretched circumference. The method also includes producing a second disposable absorbent article of size n-1 substantially equivalent in design to the first disposable absorbent article, the second disposable absorbent article including no wetness indicator and having a size n-1 longitudinal length and a waist opening with a size n-1 unstretched circumference. The method also includes producing a third disposable absorbent article of size n including a wetness indicator and having a longitudinal length of the size n-1 longitudinal length ±5% and a waist opening unstretched circumference of the size n unstretched circumference ±5%.

Other features and aspects of the present disclosure are discussed in greater detail herein.

### BRIEF DESCRIPTION OF THE DRAWINGS

The foregoing and other features and aspects of the present disclosure and the manner of attaining them will become more apparent, and the disclosure itself will be better understood by reference to the following description, appended claims, and accompanying drawings.

FIG. 1 is a side perspective view of one aspect of an absorbent article;

FIG. 2 is a plan view of the absorbent article shown in FIG. 1 with the article in an unfastened, unfolded and laid flat condition showing the surface of the article that faces away from the wearer;

FIG. 3 is a plan view of the absorbent article shown in FIG. 1 with the article in an unfastened, unfolded and laid flat condition showing the surface of the article that faces the wearer; and

FIG. 4 is a perspective view of the absorbent article illustrated in FIG. 1 including one aspect of a wetness indicator of the present disclosure.

Repeat use of reference characters in the present specification and drawings is intended to represent the same or analogous features or elements of the present disclosure.

### DETAILED DESCRIPTION

It is to be understood by one of ordinary skill in the art that the present discussion is a description of exemplary aspects only, and is not intended as limiting the broader aspects of the present disclosure.

The present disclosure is generally directed to training systems for absorbent articles that indicate to a user when a body fluid has insulted the article. For example, in one aspect, the training system is designed to provide a tactile cue when urine is deposited in the absorbent article.

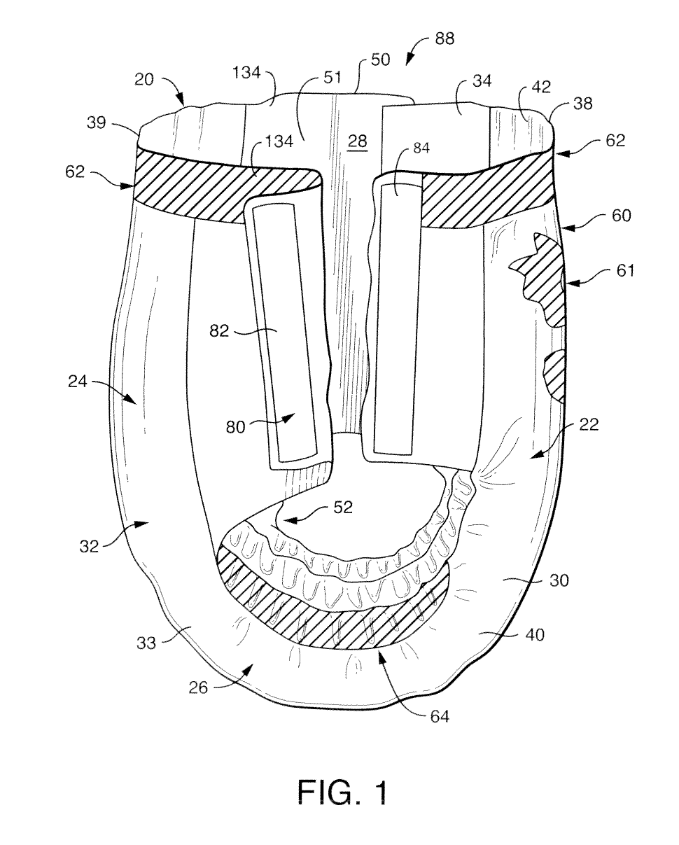

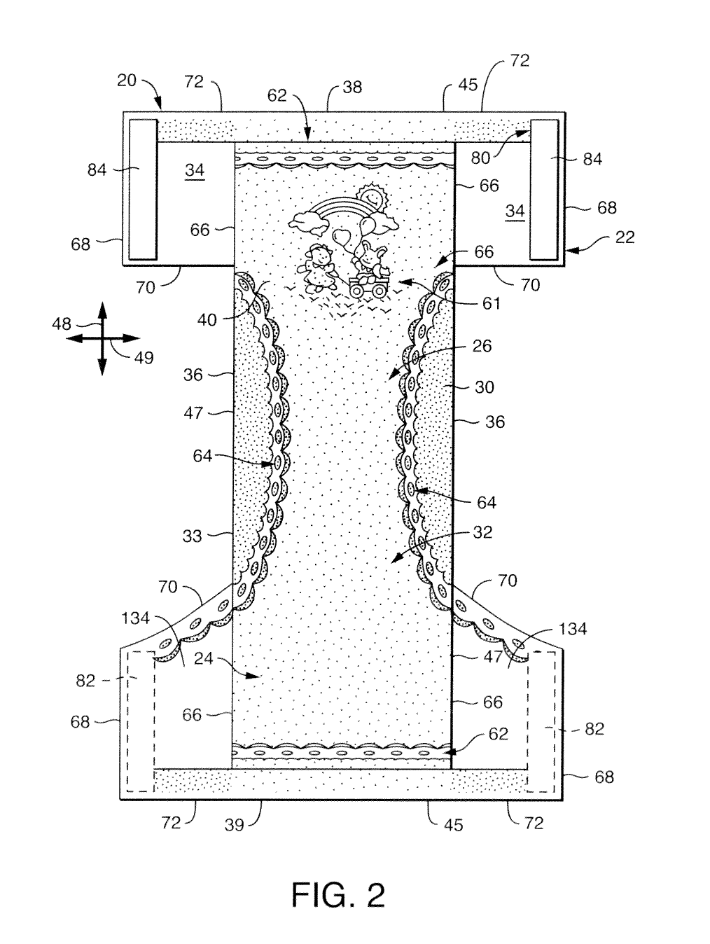

In accordance with the present disclosure, the training system can have various configurations and designs. Referring to FIGS. 1 and 2, for exemplary purposes, an absorbent article 20 that can be used in conjunction with training systems of the present disclosure is shown. The absorbent article 20 can be disposable or not. It is understood that the present disclosure is suitable for use with various other absorbent articles intended for personal wear, including but not limited to diapers, training pants, swim pants, feminine hygiene products, incontinence products, medical garments, surgical pads and bandages, other personal care or health care garments, and the like without departing from the scope of the present disclosure.

The methods and apparatus of the present disclosure can be used to make a variety of pre-fastened articles such as disposable absorbent articles including diapers, training pants, feminine hygiene products, incontinence products, medical garments, other personal care or health care garments, swim pants, athletic clothing, pants and shorts, and the like. More particularly, the methods and apparatus of the present disclosure can be used to make articles in which at least two elements of the article are connected together during the making thereof to assemble or “pre-fasten” the article. For ease of explanation, the methods and apparatus of the present disclosure are hereafter described in connection with making pre-fastened child's pants, generally indicated as 20 in FIG. 1. In particular, the methods and apparatus will be described in terms of those for making pre-fastened disposable pants as described in U.S. patent application Ser. No. 09/444,083 titled “Absorbent Articles With Refastenable Side Seams” and filed Nov. 22, 1999 (corresponding to PCT application WO 00/37009 published Jun. 29, 2000) by A. L. Fletcher et al., the disclosure of which is incorporated herein by reference. Absorbent article 20 can also be constructed using the methods and apparatus disclosed in U.S. Pat. No. 4,940,464 issued Jul. 10, 1990 to Van Gompel et al.; and U.S. Pat. No. 5,766,389 issued Jun. 16, 1998 to Brandon et al.; the disclosures of which are also incorporated herein by reference.

It should be understood that as used herein, the term “component” includes not only discrete objects, but also objects yet to be formed into discrete objects (e.g., objects yet to be severed into discrete objects from a continuous sheet or web of material), particles (e.g., superabsorbent particles or polymers), adhesives, lotions, ointments, and other substances, as well as portions or characteristics of any such components including, for example, fold lines, bond lines (e.g., ultrasonic bond lines), bonded or adhered regions, and registration marks applied to or about components for subsequent detection during a manufacturing or inspection process.

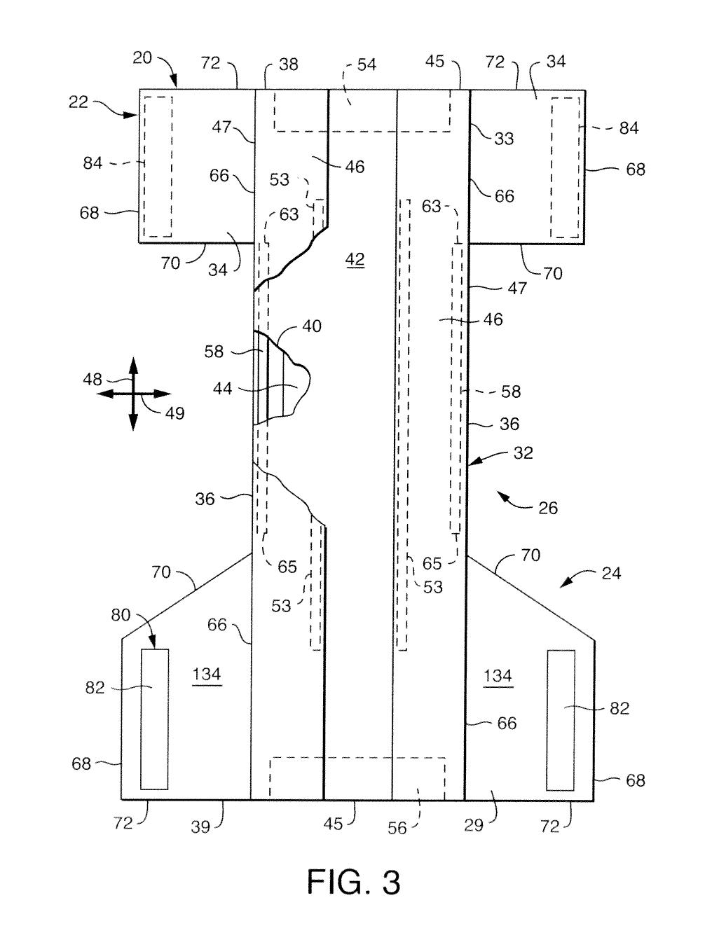

With reference now to the drawings, and in particular to FIG. 1, an absorbent article 20 is illustrated in a partially fastened condition and includes an absorbent chassis 32 having a front waist region 22, a back waist region 24, a crotch region 26 interconnecting the front and back waist regions 22,24, an inner surface 28 that is configured to contact the wearer, and an outer surface 30 opposite the inner surface and configured to contact the wearer's clothing. With additional reference to FIGS. 2 and 3, the absorbent chassis 32 also has a pair of laterally opposite side edges 36 and a pair of longitudinally opposite waist edges, respectively designated front waist edge 38 and back waist edge 39. The front waist region 22 is contiguous with the front waist edge 38, and the back waist region 24 is contiguous with the back waist edge 39.

The illustrated absorbent chassis 32 includes a composite structure 33 (FIGS. 2 and 3), which when laid flat can be rectangular or any other desired shape, and has a pair of laterally opposite front side panels 34 and a pair of laterally opposite back side panels 134 extending outwardly therefrom.

The composite structure 33 and side panels 34, 134 can include two or more separate elements, as shown in FIG. 1, or be integrally formed. Integrally formed side panels 34, 134 and composite structure 33 would include at least some common materials, such as the bodyside liner, flap composite, outer cover, other materials and/or combinations thereof, and could define a one-piece elastic, stretchable, or non-stretchable pants. The illustrated composite structure 33 includes an outer cover 40, a bodyside liner 42 (FIGS. 1 and 3) connected to the outer cover in a superposed relation, an absorbent assembly 44 (FIG. 3) disposed between the outer cover and the bodyside liner, and a pair of containment flaps 46 (FIG. 3). The illustrated composite structure 33 has opposite ends 45 (FIGS. 2 and 3) that form portions of the front and back waist edges 38 and 39, and opposite side edges 47 that form portions of the side edges 36 of the absorbent chassis 32 (FIGS. 2 and 3).

For reference, arrows 48 and 49 (FIGS. 2 and 3) depict the orientation of the longitudinal axis and the transverse or lateral axis, respectively, of the absorbent article 20.

With the absorbent article 20 in the fastened position as partially illustrated in FIG. 1, the front and back side panels 34, 134 are connected together by a fastening system 80 to define a three-dimensional pants configuration having an interior space 51, a waist opening 50 for receiving the wearer into the interior space 51 of the absorbent article 20, a pair of leg openings 52 and engagement seams 88 along which the side panels 34, 134 are connected. The interior space 51 of the absorbent article 20 is thus bounded by the absorbent chassis 32, the engagement seams 88 and the portions of the side panels 34, 134 extending on opposite sides of the engagement seams 88 (e.g., between the engagement seams 88 and the absorbent chassis 32). As used herein, the “interior space” 51 is intended to refer to the space between any two portions of a three-dimensional article that generally oppose each other. It is understood that a transverse cross-section of the article need not be closed, e.g., continuous, to define the interior space 51. For example, a two-dimensional article can be generally folded over on itself so that two portions of the article oppose each other to define an interior space of the article therebetween. Thus, the interior space 51 of the absorbent article 20 shown in FIG. 1 can be defined by the side panels 34, 134 themselves or, if the side panels are fully straightened therebetween, the interior space is defined by a combination of the side panels 34,134 and the front and back waist regions 22, 24 of the absorbent chassis 32.

The front waist region 22 includes the portion of the absorbent article 20 that, when worn, is positioned on the front of the wearer while the back waist region 24 includes the portion of the absorbent article 20 that, when worn, is positioned on the back of the wearer. The crotch region 26 of the absorbent article 20 includes the portion of the absorbent article 20 that, when worn, is positioned between the legs of the wearer and covers the lower torso of the wearer. The front and back side panels 34 and 134 include the portions of the absorbent article 20 that, when worn, are positioned on the hips of the wearer. The waist edges 38 and 39 of the absorbent chassis 32 are configured to encircle the waist of the wearer when worn and together define the waist opening 50 (FIG. 1). Portions of the side edges 36 in the crotch region 26 generally define the leg openings 52.

The absorbent chassis 32 is configured to contain and/or absorb any exudates discharged from the wearer. For example, the absorbent chassis 32 desirably although not necessarily includes the pair of containment flaps 46 that are configured to provide a barrier to the transverse flow of body exudates. A flap elastic member 53 (FIG. 3) can be operatively joined with each containment flap 46 in any suitable manner as is well known in the art. The elasticized containment flaps 46 define an unattached edge that assumes an upright configuration in at least the crotch region 26 of the absorbent article 20 to form a seal against the wearer's body. The containment flaps 46 can be located along the side edges 36 of the absorbent chassis 32, and can extend longitudinally along the entire length of the absorbent chassis 32, or can only extend partially along the length of the absorbent chassis 32. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the absorbent article 20 desirably although not necessarily includes a front waist elastic member 54, a rear waist elastic member 56, and leg elastic members 58, as are known to those skilled in the art (FIG. 3). The waist elastic members 54 and 56 can be operatively joined to the outer cover 40 and/or the bodyside liner 42 along the opposite waist edges 38 and 39, and can extend over part or all of the waist edges. The leg elastic members 58 can be operatively joined to the outer cover 40 and/or the bodyside liner 42 along the opposite side edges 36 and positioned in the crotch region 26 of the absorbent article 20. The leg elastic members 58 can be longitudinally aligned along each side edge 47 of the composite structure 33. Each leg elastic member 58 has a front terminal point 63 and a back terminal point 65, which represent the longitudinal ends of the elastic gathering caused by the leg elastic members. The front terminal points 63 can be located adjacent the longitudinally innermost parts of the front side panels 34, and the back terminal points 65 can be located adjacent the longitudinally innermost parts of the back side panels 134.

As shown in FIGS. 1 and 2, the absorbent article 20 and in particular the outer cover 40 desirably include one or more appearance-related components. Examples of appearance-related components include, but are not limited to, graphics; highlighting or emphasizing leg and waist openings in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; registering wetness indicators, temperature indicators, and the like in the product; registering a back label, or a front label, in the product; and registering written instructions at a desired location in the product.

The illustrated absorbent article 20 is designed for use by young girls and includes a registered outer cover graphic 60 (FIGS. 1 and 2). In this design, the registered graphic 60 includes a primary pictorial image 61, simulated waist ruffles 62, and simulated leg ruffles 64. The primary pictorial image 61 includes an object graphic such as a rainbow, sun, clouds, animal characters, wagon and balloons. Any suitable design can be utilized for an absorbent article 20 intended for use by young girls, so as to be aesthetically and/or functionally pleasing to them and the caregiver. The appearance-related components are desirably positioned on the absorbent article 20 at selected locations, which can be carried out using the methods disclosed in U.S. Pat. No. 5,766,389 issued Jun. 16, 1998 to Brandon et al., the entire disclosure of which is incorporated herein by reference. The primary pictorial image 61 is desirably positioned in the front waist region 22 along the longitudinal center line of the absorbent article 20.

As noted previously, the illustrated absorbent article 20 has front and back side panels 34 and 134 disposed on each side of the absorbent chassis 32. The front side panels 34 can be permanently bonded along seams 66 to the composite structure 33 of the absorbent chassis 32 in the respective front and back waist regions 22 and 24. More particularly, as seen best in FIGS. 2 and 3, the front side panels 34 can be permanently bonded to and extend transversely outward beyond the side edges 47 of the composite structure 33 in the front waist region 22, and the back side panels 134 can be permanently bonded to and extend transversely outward beyond the side edges of the composite structure in the back waist region 24. The side panels 34 and 134 can be bonded to the composite structure 33 using attachment means known to those skilled in the art such as adhesive, thermal or ultrasonic bonding. Alternatively, the side panels 34 and 134 can be formed as an integral portion of a component of the composite structure 33. For example, the side panels can include a generally wider portion of the outer cover 40, the bodyside liner 42, and/or another component of the absorbent chassis 32. The front and back side panels 34 and 134 can be permanently bonded together or be releasably connected with one another such as by the fastening system 80 of the illustrated aspect.

As best illustrated in FIGS. 2 and 3, the front and back side panels 34, 134 each have an outer edge 68 spaced laterally from the seam 66, a leg end edge 70 disposed toward the longitudinal center of the absorbent article 20, and a waist end edge 72 disposed toward a longitudinal end of the absorbent article 20. The leg end edge 70 and waist end edge 72 extend from the side edges 47 of the composite structure 33 to the outer edges 68. The leg end edges 70 of the side panels 34 and 134 form part of the side edges 36 of the absorbent chassis 32. In the back waist region 24, the leg end edges 70 are desirably although not necessarily curved and/or angled relative to the transverse axis 49 to provide greater coverage toward the back of the absorbent article 20 as compared to the front of the absorbent article 20. The waist end edges 72 are desirably parallel to the transverse axis 49. The waist end edges 72 of the front side panels 34 form part of the front waist edge 38 of the absorbent chassis 32, and the waist end edges 72 of the back side panels 134 form part of the back waist edge 39 of the absorbent chassis. The waist end edges 72 are generally aligned or co-linear with the front and back waist edges 38, 39.

In particular aspects for improved fit and appearance, the side panels 34, 134 desirably have an average length measured parallel to the longitudinal axis 48 that is about 15 percent or greater, and particularly about 25 percent or greater, of the overall length of the absorbent article 20, also measured parallel to the longitudinal axis 48. For example, in absorbent article 20 having an overall length of about 54 centimeters, the side panels 34, 134 desirably have an average length of about 10 centimeters or greater, such as about 15 centimeters. While each of the side panels 34, 134 extends from the waist opening 50 to one of the leg openings 52, the illustrated back side panels 134 have a continually decreasing length dimension moving from the seam 66 to the outer edge 68, as is best shown in FIGS. 2 and 3.

Each of the side panels 34, 134 can include one or more individual, distinct pieces of material. In particular aspects, for example, each side panel 34, 134 can include first and second side panel portions that are joined at a seam, or can include a single piece of material that is folded over upon itself (not shown).

The side panels 34, 134 desirably although not necessarily include an elastic material capable of stretching in a direction generally parallel to the transverse axis 49 of the absorbent article 20. Suitable elastic materials, as well as one process of incorporating elastic side panels into an absorbent article 20, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. An alternative elastic material is described below. In particular aspects, the elastic material includes a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; and European Patent Application No. EP 0 217 032 published on Apr. 8, 1987 in the names of Taylor et al.; all of which are incorporated herein by reference. Alternatively, the side panel material can include other woven or nonwoven materials, such as those described above as being suitable for the outer cover 40 or bodyside liner 42, mechanically pre-strained composites, or stretchable but inelastic materials.

Further detail with respect to elastic laminates of the present disclosure can be found in co-pending U.S. Patent Publication No. 2008/0095978 entitled “Nonwoven Composite Containing an Apertured Elastic Film,” which is incorporated herein by reference to the extent it does not conflict herewith.

Absorbent article 20 can have the side panels 34, 134 affixed to each other in for securing the absorbent article 20 about the waist of the wearer. The side panels 34, 134 can be affixed by bonding, mechanical fasteners, or any other suitable method, and can be affixed permanently, in a tearable manner, or in a refastenable manner. The illustrated absorbent article 20 includes the fastening system 80 for refastenably securing the absorbent article 20 about the waist of the wearer. The illustrated fastening system 80 includes first fastening components 82 adapted for refastenable engagement to corresponding second fastening components 84. In one aspect, one surface of each of the first fastening components 82, 84 includes a plurality of engaging elements that project from that surface. The engaging elements of the first fastening components 82 are adapted to repeatedly engage and disengage engaging elements of the second fastening components 84.

The fastening components 82, 84 can include separate elements bonded to the side panels 134, 34, or they can be integrally formed with the side panels. Thus, unless otherwise specified, the term “fastening component” includes separate components that function as fasteners, and regions of materials such as the side panels 34, 134 that function as fasteners. Moreover, a single material can define multiple fastening components to the extent that different regions of the material function as separate fasteners. The fastening components 82, 84 can be located on the side panels 134, 34, between the side panels such as on the absorbent chassis, or a combination of the two.

The design of the absorbent article 20 of the present disclosure results in a ratio of product length to circumference that is significantly differentiated from other absorbent products, and also results in significantly better performance as more wearers feel a tactile sensation, be it cooling, heating, fizzing, swelling, or some combination of these, than in other absorbent products.

It is significant to note that this solution is particularly important for training products where wear time is shorter and the products are not worn overnight. As the rise is decreased, the absorbent articles can leak more when worn for an extended period due to the decreased bucketing. Manufacturers typically attempt to increase the period over which an article can be worn to, for example, extend a training pant line to overnight use. As a result, the shorter rise/decreased bucketing of the absorbent article of the present disclosure runs contrary to typical thinking and products.

The present disclosure is directed to an absorbent article 20 having optimized parameters. The principles of the present disclosure can be incorporated into any suitable pant-like disposable absorbent article 20. Examples of such suitable articles include diapers, diaper pants, training pants, incontinence products, other personal care or health care garments, including medical garments, or the like. As used herein, the term incontinence products includes absorbent underwear for children, absorbent garments for children or young adults with special needs such as autistic children or others with bladder/bowel control problems as a result of physical disabilities, as well as absorbent articles for incontinent older adults. For ease of explanation, the description hereafter will be in terms of a child's training pant.

The term “training pant,” as used herein, refers to a pant-style absorbent article having either permanently bonded side seams or refastenable side seams that are packaged and sold in a pre-fastened, ready-to-wear position. In other words, the training pant is provided with a continuous waist circumference when the absorbent article is delivered to the consumer with any pre-determined fastening determined by the manufacturer. Thus, the training pant has an automatic fit, as opposed to an adjustable fit, with a pertinent waist circumference determined by the manufacturer such that the training pant is packaged by the manufacturer in a user-friendly mode wherein a wearer can put the absorbent article on without having to manually adjust any fastening devices.

Training pants and other pant-like absorbent articles are typically marketed in sizes that correspond to an intended wearer's weight. For purposes of describing the absorbent article 20 of the present disclosure in terms of size independent of the intended wearer, the absorbent article size is described in terms of the circumference of the waist opening 50. A method for determining the waist circumference (at 500 grams tension) is described in detail below. In the absorbent article 20 of the present disclosure, the circumference of the waist opening 50 can be in a range of between about 450 mm and about 750 mm, or between about 500 mm and about 700 mm.

The absorbent article 20 of the disclosure has been configured in a manner that provides desirable product features and the resulting product has been found to have a certain relationship between the longitudinal length and the waist circumference. The longitudinal length of the absorbent article 20 can be measured according to the method described in detail below. In the absorbent article 20 of the present disclosure, the longitudinal length of the absorbent article can be in a range of between about 350 mm and about 650 mm, or between about 375 mm and about 600 mm, or between about 400 mm and about 575 mm.

The resulting absorbent article has a longitudinal length that is proportional to the unstretched circumference of the waist opening by a ratio less than 0.82, by a ratio less than 0.80, or by a ratio less than 0.78.

In an alternative aspect of the present disclosure, the absorbent article can be sized such that the CD circumference is generally equivalent to that of non-wetness indicating absorbent article of similar size, but the MD length is generally equivalent to that of non-wetness indicating absorbent articles of the next smaller size. In other words, where a first disposable absorbent article of size n including no wetness indicator has a size n longitudinal length and a waist opening with a size n unstretched circumference; and where a second disposable absorbent article of size n-1 that is substantially equivalent in design to the first disposable absorbent article also includes no wetness indicator and has a size n-1 longitudinal length and a waist opening with a size n-1 unstretched circumference, the absorbent article of the present disclosure includes a wetness indicator and has a longitudinal length of the size n-1 longitudinal length ±5% and a waist opening unstretched circumference of the size n unstretched circumference ±5%. In this aspect, the absorbent article of the present disclosure can include a temperature change agent, a swellable wetness indicator, or both.

The absorbent article 20 has a length dimension measured between the front and back end edges 38, 39 along the longitudinal axis 48.

A suitable method for determining the longitudinal length of the absorbent article 20 is described in more detail in co-assigned U.S. Patent Application Publication No. US 2003/0181883 A1 to Olson et al., entitled “Garment-Like Absorbent Article,” which is incorporated herein by reference to the extent it does not conflict herewith. The method includes hanging the absorbent article 20 vertically adjacent a flat, vertical surface. Prior to hanging, the absorbent article 20 is opened by cutting or opening any side seams. Any elastic components that run the length of the chassis (such as leg elastics or elastics within containment flaps) are severed at least once per inch along their entire length. The absorbent article 20 is hung with the back region 24 above the front region 22 and with the surface intended to face the wearer's outer garments during use positioned toward the flat, vertical surface. The top end margin of the absorbent article 20 is held horizontal with two clamps, the inner edges of which are spaced 3.5 inches (8.9 cm) apart. The clamps are positioned if possible to avoid any absorbent within the absorbent article 20, and are symmetrically disposed with respect to the longitudinal centerline/axis of the absorbent article 20. Any waist elastic present in the absorbent article 20 is not stretched prior to securing the clamps.

The lower end of the hanging absorbent article 20 (front waistband region) is clamped with a jig weighing 250 g. The jig possesses two clamp units (medium size, Bulldog clips, 2⅛ inch) attached to a tie rod (¼-inch-20×12 inches, coarse thread, zinc plated), the clamps symmetrically placed with respect to the longitudinal centerline of the absorbent article 20, with a spacing between internal edges of the clamps of 3.5 inches (8.9 cm), with a ¼-inch nut placed at the inner and outer edges of each clamp to hold the clamps in place. One (capped) bottle (1-ounce plastic screw cap bottle, such as NALGENE brand) is attached to each clamp with a piece of string. The assembly is placed on a laboratory balance and lead shot (No. 5 chilled lead shot, such as LAWRENCE brand) is added to each bottle (in equal amounts) until the total weight of the jig is as close to 250 grams as possible. The jig is attached to the lower end of the hanging absorbent article 20, as mentioned above.

For a typical absorbent article 20, a load of 250 g is appropriate. The elongate length is then determined by measuring the distance between the front and back end edges 38, 39 along the longitudinal centerline/axis 48, between the clamps. Five specimens of each code are analyzed, and the results for each code are averaged.

A single-cycle tension bench test is used to measure waistband circumference of a test pant. The absorbent article 20 is tested in the state in which it is provided to the consumer, intended for immediate donning. The absorbent article 20 is opened and laid flat on a flat surface. A linear measurement of each waist area is taken with the pant in an unstretched state. The two waist area measurements are then added to determine the CD circumference measurement of the pant.

Further information with respect to reduced rise in disposable absorbent articles can be found in the previously-described U.S. Patent Application Publication No. US 2003/0181883 A1 to Olson et al., which is incorporated herein by reference to the extent it does not conflict herewith.

In a specific example, typical HUGGIES PULL UPS training pants of size 3 T-4 T are designed to fit a wearer of 32-40 pounds and have a 495 mm MD pant length and a 205 mm width (CD w/o side panels). This product has 90 mm of side panels on each side for a total un-stretched circumference of 590 mm. This gives a ratio of 0.83 for MD length to CD circumference. PAMPERS EASY-UPS training pants of size 5 are designed to fit a wearer of 30-40 pounds and have a 480 mm MD length and a 165 mm CD width plus 240 mm of stretch material for a total CD unstretched circumference of 570 mm. This gives a ratio of 0.84 for MD length to CD circumference. An absorbent article of the present disclosure designed for the 3 T-4 T fit range can have an MD length of 451 mm and a CD circumference of 590 mm for a ratio of 0.76.

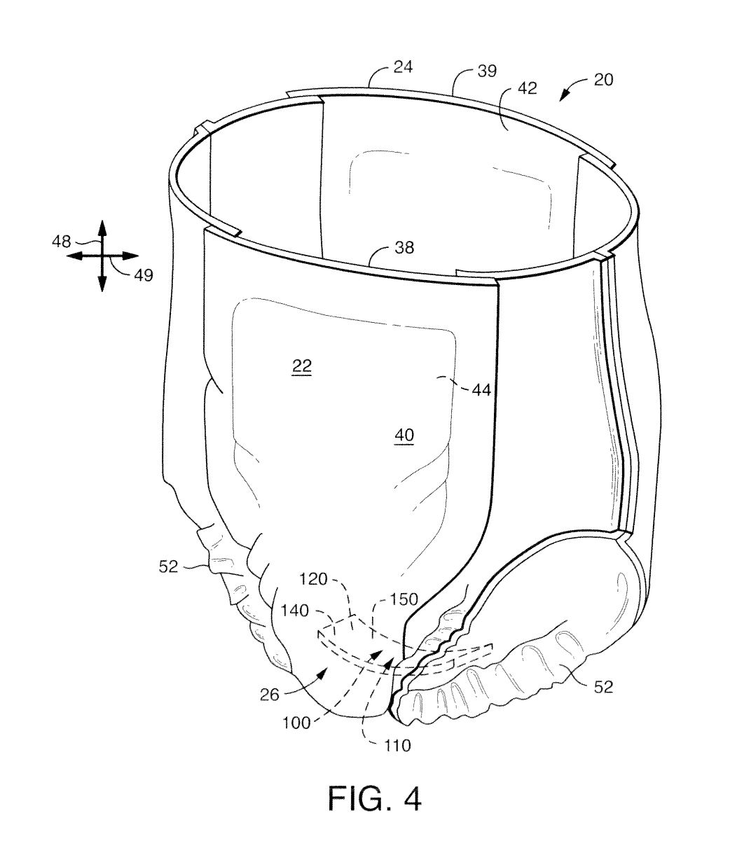

As illustrated in FIG. 4, a wetness indicator for alerting a wearer to urination, generally designated by 100, is positioned generally between the leg openings 52 of the absorbent article 20. The wetness indicator 100 can include one or both of a swell element 110 and physical sensation element 120. The wetness indicator 100 is generally positioned between the absorbent assembly 44 and the wearer. The swell element 110 and the physical sensation element 120 can be positioned in an overlying relationship on the wearer-facing surface of the liner 42, or between the liner 42 and the absorbent assembly 44. In another aspect of the present disclosure, the swell element 110 can be positioned between the liner 42 and the absorbent assembly 44, and the physical sensation element 120 can be positioned on the wearer-facing surface of the liner 42. In one aspect of the present disclosure, the physical sensation element 120 can be a temperature element 140.

A swell element 110 is positioned generally between the leg openings 52 of the absorbent article 20. It should be understood that the swell element 110 can be positioned elsewhere than in the crotch region 26 without departing from the scope of the present disclosure. It is envisioned that the position of swell element 110 within the crotch region 26 can be different depending on whether the absorbent article 20 is intended to be worn by girls or boys. As will be discussed in greater detail below, the swell element 110 is configured to absorb liquid, such as urine. Therefore, placement in a more forward position of the crotch region 26 can be appropriate for boys, and placement in a more central position between the legs can be appropriate for girls. Before absorption of such liquid, the swell element 110 is pliable and virtually imperceptible to the wearer, as will be discussed in greater detail below.

The wetness indicator 100 can also be used in conjunction with other garments and/or absorbent articles, such as underwear, diapers, and washable or reusable absorbent articles such as woven training pants, absorbent swim pants, plastic training pants, and the like. Further, although discussed primarily in the context of toilet training for children, it should be understood that the present disclosure is applicable to adult personal care products such as absorbent incontinence undergarments and the like. The wetness indicator 100 can either be built directly into the article during manufacture or can be formed independently and attached to any of the aforementioned articles by the consumer. If the wetness indicator 100 is built directly into the article during manufacture, the wetness indicator 100 can be optionally releasably attached so the consumer can remove the wetness indicator if desired.

In one aspect, the swell element 110 is generally elongate, and remains elongate upon absorption of a preselected amount of liquid, as will be discussed in greater detail below. Although other orientations are contemplated as being within the scope of the disclosure, the swell element 110 of one aspect is oriented generally laterally within the absorbent article 20, such that the longest dimension of the swell element 110 lies substantially parallel to a lateral axis 49 of the absorbent article 20 that extends between the leg openings 52. The periphery of the swell element 110 of this aspect is also generally rectangular in cross-section before absorption of the preselected amount of liquid, and after absorption, the swell element 110 can become generally rounded. It is also contemplated that the swell element 110 of the present disclosure can be formed in a shape other than a rectangle, without departing from the scope of the present disclosure.

The swell element 110 includes an absorbent body. It is desirable that the absorbent body be formed from a high absorbency under load (AUL) material. Although the absorbent body can be made from other materials without departing from the scope of the present disclosure, in one aspect the body is an Ultra Thin Absorbent (UTA) material comprising about fifty percent superabsorbent material and about fifty percent wood pulp and having a weight of about 225 grams per square meter (gsm) at a thickness of about 0.65 millimeter. One such UTA material can be produced using an online process such as described in U.S. application Ser. No. 09/939,061, entitled “Thin, High Capacity Absorbent Structure and Method for Producing Same,” filed Aug. 24, 2001, by Sawyer, et al., which claims priority from U.S. Provisional Application No. 60/256,016, filed Dec. 20, 2000, both of which are hereby incorporated by reference. In addition, U.S. Pat. No. 5,147,343 (Kellenberger) and U.S. Pat. No. 5,601,542 (Melius et al.) describe other processes for producing UTA material. Alternate materials include NOVATHIN absorbent material, available from EAM Corporation of Jessup, Ga., and KINOCLOTH superabsorbent sheets, manufactured by Oji Kinocloth Co., Ltd.

When liquid contacts the absorbent body, the liquid is absorbed by the absorbent body. Capillary, osmotic, and absorptive forces draw the liquid into the absorbent body. This absorption process helps draw liquid away from the skin of the wearer. As the absorbent body absorbs the liquid, the absorbent body begins swelling. When dry, the swell element 110 is generally soft, pliable and cloth-like with a stiffness similar to the other portions of the absorbent article 20, making the presence of the swell element 110 generally imperceptible to the wearer. The pliable swell element 110 allows the thighs to move freely and easily compress the swell element 110.

Once liquid is present in the absorbent article 20 and the absorbent body begins absorbing liquid, the absorbent body swells. Because the unrestrained saturated volume of the absorbent body is greater than the dry volume of the absorbent body, the swell element 110 increases in thickness. As more liquid is absorbed by the body, the swell element 110 becomes thicker. Once the swell element 110 absorbs the preselected amount of liquid, the wetness indicator reaches a second thickness greater than the first thickness. Such a thickness provides a resistance to bending that can be readily perceived by the wearer.

The swell element 110 can assist in indicating wetness in two manners. The increase in thickness of the swell element 110 due to liquid absorption can result in increased pressure within the absorbent article 20 and thus increased pressure on the wearer, giving a tactile wetness indication. The increase in thickness can also cause the physical sensation element 120 to rise, becoming closer to the wearer's skin for increased contact with the physical sensation element 120.

Although the swell element 110 can have other thicknesses without departing from the scope of the present disclosure, in one configuration, the swell element 110 has a second thickness about eleven times greater, on average, than its first thickness. In another configuration, the second thickness is about fourteen times greater, on average, than the first thickness. An effective swell element 110 should have a second thickness at least about three times greater than its first thickness.

For example, a swell element 110 with an average dry length of about 11.5 cm, an average dry width of about 2.0 cm and an average dry thickness of about 0.5 cm and composed of the materials described herein was used. Although the second thickness can be measured upon absorption of a different amount of liquid without departing from the scope of the present disclosure, in one aspect the second thickness is measured upon absorption of about 33 grams of urine. However, those skilled in the art will appreciate that the amount of urine absorbed by the absorbent body in actual use varies depending upon the size of the swell element 110 and the materials used. In other words, the thickness can be different in actual use.

Absorption of the preselected amount of liquid expands the swell element 110, which lifts the liner 42 toward the skin of the wearer.

Furthermore, it is important that the preselected amount of liquid remain within the absorbent body even under outside pressure, such as applied by the leg of the wearer. If wearer movement forces liquid from the absorbent body, the ability of the wearer to perceive a thickened swell element 110 decreases. Because the wearer can be active at the time of urination, the ability of the swell element 110 to resist outside forces is important. If the amount of liquid in the absorbent body remains above the preselected amount, the thickness of the swell element 110 can be maintained continually for a sustained and perceptible indication that urination has occurred. Forming the absorbent body from a high AUL material resists releasing liquid, even under pressure.

Further information with respect to swelling and stiffening wetness indicators can be found in co-assigned U.S. Patent Application Publication No. US 2003/0125682 A1, entitled “Wetness Indicator for Alerting a Wearer to Urination,” which is incorporated herein by reference to the extent it does not conflict herewith.

With particular reference now to FIG. 4, the physical sensation element 120 is suitably disposed between the bodyside liner 42 and the outer cover 40 so that the wetness indicator is substantially imperceptible to the wearer prior to the first insult of the absorbent article 20 by liquid body exudates, e.g., in the case of training pants, urine. The physical sensation element 120 is longitudinally positioned in the crotch region 26 of the absorbent article 20 between the leg openings 52 (FIG. 1) thereof. However, it is contemplated that the longitudinal position of the physical sensation element 120 within the crotch region 26 can be dependent on whether the absorbent article 20 is to be worn by a boy or a girl. For example, placement of the physical sensation element 120 in a more forward location within the crotch region 26 can be appropriate for boys, while placement in a more central location within the crotch region can be more appropriate for girls. It is also understood that the physical sensation element 120 can be positioned other than in the crotch region 26 without departing from the scope of the present disclosure, as long as the physical sensation element 120 is suitably positioned so as to become wet and perceptible by a wearer upon insult of the absorbent article 20 by liquid body exudates.

Also, the physical sensation element 120 can be disposed other than between the liner 42 and the outer cover 40, e.g., it can be disposed on the liner 42 for direct contact with the wearer's skin and remain within the scope of this disclosure. In such an aspect, the physical sensation element 120 can be attached to the liner 42, such as by being adhesively attached or bonded thereto, or it can be releasably secured to the liner 42 to permit the caregiver to position the physical sensation element 120 on the liner 42 in a desired longitudinal position depending on whether the absorbent article 20 is to be worn by a boy or a girl.

In one aspect of the present disclosure, the physical sensation element 120 is positioned to generally overlie the swelling element 110, and can be attached thereto by any suitable means. In this aspect, the physical sensation element 120 can be attached to the liner 42, and the swelling element 110 can be attached to the absorbent assembly 44 by any suitable means.

While a single physical sensation element 120 is shown in the illustrated aspect, it is contemplated that additional physical sensation elements 120 can be used to further enhance the signal to the wearer. For example, additional physical sensation elements 120 can be necessary for larger or older children with larger legs for whom the resistive force provided by a single physical sensation element 120 is generally insufficient to alert the wearer to his or her urination. A pair of physical sensation elements 120 can also be used in a configuration wherein one physical sensation element 120 is positioned longitudinally where it is more likely to become wet upon urination by boys and the other physical sensation element 120 is positioned longitudinally where it is more likely to become wet upon urination by girls, thereby accounting for differences between the target wetting areas of boys and girls.

In one aspect of the present disclosure, the physical sensation element 120 of the illustrated aspect suitably extends across the majority of the crotch region 26 of the absorbent article 20, having a length substantially equal to the width of the absorbent assembly 44. As an example of relative dimensions, the crotch region 26 of the absorbent article 20 of FIG. 4 has a width of approximately 109 mm, the absorbent structure 44 has a width at the crotch region 26 of about 89 mm, and the physical sensation element 120 has a length of about 89 mm and a width of about 51 mm. However, it is understood that the physical sensation element 120 can be shorter or longer than the width of the absorbent structure 44. Also, the longitudinal extent of the physical sensation element 120 can be greater than or equal to the transverse extent thereof, or it can be less than that shown in FIG. 4, without departing from the scope of this disclosure.

The thickness of the physical sensation element 120 when dry is suitably in the range of about 2 mm to about 15 mm, and more suitably in the range of about 3 mm to about 9 mm.

The physical sensation element 120 can include a co-form material as is known in the art or can include a physical sensation laminate. If in the form of a physical sensation laminate, the physical sensation element 120 can include a physical sensation change agent and a polymeric buffering film. The purpose of the physical sensation change agent is to provide the wearer with a perceptible sensation when a fluid insult is occurring and/or has occurred. The physical sensation agent is preferably in the form of a solid. As used in this context, the term “solid” as it refers to temperature change agents can include particles, flakes, fibers, agglomerates, granules, powders, spheres, pulverized materials, tablets or the like, as well as combinations thereof. The solids can have any desired shape such as, for example, cubic, rod-like, polyhedral, spherical or semi-spherical, rounded or semi-rounded, angular, irregular, etc.

Incorporating the physical sensation change agent into a polymeric buffering film including a water-soluble, and optionally water-swellable, base polymer, and optional additives, can delay the time scale to start the sensation effect and/or to maximize the sensation effect once a liquid insult has begun.

In some particular aspects, water-soluble polymers suitable for the polymeric buffering films include, but are not limited to, polyethylene oxide (PEO), polyethylene glycol (PEG), polyvinyl alcohol (PVOH), or the like, and combinations thereof. In addition, ethylene vinyl acetate copolymer (EVA) can be optionally utilized to control the water solubility of the polymer. In desirable aspects, the polymeric buffering film is present in the form of a carrying substrate. Thus, the polymeric buffering film provides a carrier film layer for the physical sensation change agent. Accordingly, the combination of the polymeric buffering film and the physical sensation change agent provide the physical sensation element 120 of the present disclosure.

In some aspects, the polymeric buffering film can include other optional additives such as a pH adjuster and/or a plasticizer. For example, suitable plasticizers include, but are not limited to, polyhydroxy organic compounds such as glycerin and low molecular weight polyolefinic glycols such as polyethylene glycol (PEG) of molecular weight ranges from about 200 to about 10,000.

To construct the physical sensation element 120 of the present disclosure, the physical sensation change agent is incorporated with the polymeric buffering film. Incorporating the physical sensation change agent with the polymeric buffering film can be accomplished by any suitable method. For example, the physical sensation change agent can be added directly into the polymeric buffering film prior to the film solidifying, such as through an extrusion process, or by melting at least portions of the film into a molten state and then adding the physical sensation change agent to the molten portion. In another example, the physical sensation change agent can be attached to the polymeric buffering film via an adhesive. In still another example, at least portions of the polymeric buffering film can be melted into a molten state and the physical sensation change agent can be sprayed onto the film. In still another example, the physical sensation change agent can be compressed into the polymeric buffering film. Other methods for incorporating the physical sensation change agent with the polymeric buffering film will be readily apparent to those skilled in the art.

Additional features of a physical sensation laminate include those described in co-pending U.S. patent application Ser. No. ______, entitled “Absorbent Article Having Improved Signal Member,” which is incorporated herein by reference to the extent it does not conflict herewith.

Still referring to FIG. 4, the absorbent structure 44 extends longitudinally from generally within the front waist region 22 of the absorbent article 20 through the crotch region 26 to generally within the back waist region 24 of the absorbent article 20. The physical sensation element 120 suitably overlies the absorbent assembly 44 within the crotch region 26. However, it is contemplated that the physical sensation element 120 can either underlie the absorbent assembly 44 or it can be enclosed within the absorbent assembly 44 (e.g., with a portion of the absorbent assembly 44 overlaying the wetness indicator 100 and a portion of the absorbent assembly 44 underlying the wetness indicator 100). The physical sensation element 120 can be attached to the absorbent structure 44, and/or to the bodyside liner 42 (or to the outer cover 40 where the wetness indicator 100 underlies the absorbent assembly 44), such as by ultrasonic bonding, adhesives, thermal bonds, or other suitable attachment techniques.

In use, the physical sensation element 120 is generally soft, pliable, and cloth-like when dry and has a thickness generally similar to that of other portions of the absorbent article 20, and more particularly the absorbent structure 44, making the presence of the wetness indicator 100 generally imperceptible to the wearer prior to urination. The pliable physical sensation element 120 allows the thighs of the wearer to move freely and to readily compress the physical sensation element 120 during normal movements. Upon the first insult of liquid body exudates, such as urine, the liquid permeates through the bodyside liner 42.

The permeability and wettability of the bodyside liner 42 are suitably balanced so that substantially all of the first liquid insult of the article does not immediately pass through the liner into the absorbent assembly 44. When the wearer is sitting or standing, this delay in penetration of the bodyside liner 42 results in the force of gravity drawing the liquid down into the crotch region 26 of the absorbent article 20 toward the physical sensation element 120 rather than being distributed to other portions of the absorbent assembly 44. This channeling mechanism is particularly useful in articles such as absorbent article 20 worn by boys because urine usually exits the urethra further from the crotch region 26 (where the physical sensation element 120 is positioned) than for girls. Thus, a greater amount of urine from the first insult reaches the physical sensation element 120 so that the child gets tactile feedback after the first insult. This helps the child more closely associate the cause (urination) with the effect (uncomfortable absorbent article 20).

As stated above, the physical sensation element 120 can include a temperature element 140. The temperature element 140 includes a temperature change agent that facilitates a tactile signal (e.g., a hot or cold sensation) against the wearer's skin to further alert the wearer that urination has occurred. While the temperature change agent can be used with any of the aspects described herein as having an absorbent assembly 44 with an absorbent capacity gradient therein, it is understood that the temperature change agent can be disposed in an absorbent article 20 in which the absorbent assembly 44 is of generally uniform absorbent capacity or has an absorbent capacity other than that described previously herein. The temperature change can be caused by either an absorption or a release of heat by the temperature change agent to change the temperature of the urine and hence surrounding components of the absorbent article 20 to a temperature noticeable to the wearer. For example, an absorption of heat by the temperature change agent will provide a cool sensation against the wearer's skin while a release of heat by the temperature change agent will provide a warm sensation (e.g., warmer than the wearer's skin temperature) against the wearer's skin.

The temperature change agent is suitably responsive to contact with an aqueous solution, such as urine, to either absorb or release heat. The mechanism by which this is accomplished can be the dissolution of the temperature change agent in the aqueous solution, the swelling of the agent in the aqueous solution and/or the reaction of the agent in the aqueous solution. In particular aspects, the temperature change agent is suitably in the form of particles that have a substantial energy difference between a dissolved state and a crystalline state, so that energy in the form of heat is absorbed or released to the environment upon contact with an aqueous solution such as urine. In other aspects, the temperature change agent releases or absorbs energy during swelling or reacting of the temperature change agent with an aqueous solution such as urine.

While a wide variety of temperature change agents can result in a temperature change in response to contact with an aqueous solution, the selection of a particular temperature change agent and the determination of the amount to be used are based at least in part on the desired temperature change to be experienced by the wearer. For example, the temperature change agent suitably provides a temperature change between the dry skin temperature prior to the urine insult and the skin temperature after the urine insult between about 5 and about 25 degrees Fahrenheit (° F.) (2.8° C.-13.8° C.). A further example of a suitable temperature change is a reduction in the urine temperature from an initial, body core temperature of about 98° F. to 100° F. (37° C.-38° C.) to a cooled temperature of about 55° F. to 75° F. (13° C.-24° C.), however, lowering the urine temperature to below 13° C. can also be suitable in the temperature element 140. To achieve this result, the temperature change agent, the amount used, and the location of the agent within the absorbent article 20 is selected so that the potential total energy change is from about 6 to about 30 calories per square centimeter (cal/cm²), which can represent either a potential total energy release of from about 6 to about 30 cal/cm² or a potential total energy absorption of from about 6 to about 30 cal/cm². More suitably, the temperature change agent, the amount used, and the location of the temperature change agent within the absorbent article 20 is selected so that the potential total energy change is from about 12 to about 24 cal/cm², and more particularly about 18 cal/cm².

As referenced above, temperature change agents suitable for use in the absorbent article 20 include those that dissolve in an aqueous solution. The solubility of such temperature change agents is suitably in the range of about 0.1 to about 3 grams of water (H₂O) per gram of agent (g/g), and more particularly from about 0.1 to about 2 g/g.

In one particular aspect, the temperature change agent suitably includes an endothermic material, which as used herein refers to any material that absorbs heat upon contact with an aqueous solution to provide a negative temperature change (e.g., a cooling of the aqueous solution). By way of illustration, suitable endothermic materials that absorb heat during dissolution upon contact with an aqueous solution include without limitation salt hydrates such as sodium acetate (H₂O), sodium carbonate (10H₂O), sodium sulfate (10H₂O), sodium thiosulfate (5H₂O), and sodium phosphate (10H₂O); anhydrous salts, such as ammonium nitrate, potassium nitrate, ammonium chloride, potassium chloride, sodium bromide, magnesium chloride, calcium chloride (6H₂O), magnesium sulfate and sodium nitrate; organic compounds, such as urea, xylitol and other sugars, and the like.

In another aspect, the temperature change agent includes a material that releases heat during dissolution, including, without limitation, aluminum chloride, aluminum sulfate, potassium aluminum sulfate, and the like.

The temperature change agent can also, or can instead, include a material that absorbs or releases heat during swelling upon contact with aqueous solution. By way of illustration, one suitable material that releases heat during such swelling is a lightly cross-linked partially neutralized polyacrylic acid. Alternatively, or additionally, the temperature change agent can include a material that absorbs or releases heat upon reaction with an aqueous solution. Additional examples of suitable temperature change agents are further described in co-assigned U.S. Pat. No. 5,702,376 entitled “Toilet Training Aid Providing A Temperature And Dimensional Change Sensation,” issued Dec. 30, 1997 to Glaug et al.

The temperature change agent is in the form of endothermic material particles, such as urea particles, disposed within the temperature element 140. More particularly, the particles are disposed on and/or within the temperature element 140. The temperature element 140 thereby contains or otherwise at least inhibits movement of the temperature change agent particles within the temperature element 140 when the temperature element 140 is dry. As an example, a suitable amount of urea particles enclosed within the enclosure of the temperature element 140 of FIG. 10 is in the range of about 5 to about 70 grams weight of urea particles. The selection of urea particles in this amount results in a drop in temperature on the surface of the temperature element 140 to about 65° F. to 75° F. (18° C.-24° C.) at about 30 to 60 seconds following the urine void or insult with saline.

In use, as urine passes onto or into the temperature element 140 of FIG. 4, the urine comes into contact with and dissolves the temperature change agent during or shortly after urination. The temperature change agent absorbs heat from the urine upon dissolution and the cooled urine is then absorbed into the temperature element 140. The cooled temperature element 140 acts in the manner of a heat sink in thermally conductive contact with the wearer's skin (e.g., via the liner 42 and/or containment flaps of the absorbent article 20) to thereby draw heat from the wearer and provide a cool sensation to the wearer's skin. Positioning the cooled temperature element 140 in thermally conductive contact with the wearer for a significant duration of time allows the temperature change resulting from the temperature change agent to be more easily noticed by the wearer.

The swelling and stiffening of the wetness indicator 100 thus facilitates a more direct thermally conductive contact between the cooled temperature element 140 and the wearer's skin. Where air space exists between the article and the wearer's skin, as could happen in prior articles that rely on temperature change as an indicator of wetness, conductive heat transfer does not occur or is otherwise substantially reduced. The effects of convection and radiation are relatively insignificant. Use of the stiffening wetness indicator 100 to hold the cooled sensation against the wearer's skin reduces the ability of the wearer to escape thermally conductive contact with the heat sink (e.g., the cooled and stiffened wetness indicator 100) by shifting their body position.

Further information with respect to temperature change wetness indicators can be found in co-assigned U.S. Patent Application Publication No. 2004/0254549 A1, entitled “Personal Wear Article with Wetness Indicator,” which is incorporated herein by reference to the extent it does not conflict herewith.

In other aspects of the present disclosure, the physical sensation element 120 can include a fizzing element 150. Applying the appropriate substance(s) directly to or within the bodyside liner 42, absorbent assembly 44, surge layer, or swelling element 110 will result in a foaming or fizzing physical sensation upon insult with urine or other body exudates. In essence, the substances described below can be used to produce a foaming or fizzing sensation directly to the wearer's skin.

As noted above, the fizzing element 150 includes a system capable of generating a gas upon being wetted. The gas that is produced in the fizzing element 150 upon wetting interacts with one or more surfactants, which are discussed below, and produces foam and causes it to press against the skin of the wearer to alert the wearer that the absorbent article 20 has been insulted.

In one aspect, the system capable of generating gas upon being wetted includes at least one acid and at least one base. The acid and base react together upon being wetted to produce a gas that can be, for example, carbon dioxide gas. The exact gas produced by the gas producing system is not critical, so long as the gas produced is substantially non-harmful to the skin of the wearer.

In another aspect, the system capable of generating a gas upon being wetted includes a urine-or-other-body-exudates-soluble effervescent solid material produced in such a manner such that a pressurized gas is trapped within cells located in the solid material. When the solid material having pressurized gas-containing cells is contacted with urine or other body exudates, the solid material begins to dissolve and the pressurized gas is released from the cells during dissolution of the solid material. This gas can interact with the surfactant to produce foam and bubbles.

In this aspect, the soluble effervescent solid material can include a sugar compound such as a mono-saccharide, di-saccharide, or poly-saccharide that has been infused with a gas that is substantially non-reactive with human skin. Suitable gases for infusion into a solid material include, for example, carbon dioxide, air, nitrogen, argon, helium, other substantially inert gases, and combinations thereof. Specific examples of saccharides that can be used in accordance with the present disclosure include glucose, fructose, sucrose, lactose, maltose, dextrin, cyclodextrin, and the like, alone or in combination. Also, a mixture of sucrose with corn syrup (containing glucose, maltose, and dextrin) can be used in accordance with this aspect of the present disclosure to produce a gas-containing effervescent agent. Other examples of compounds that are capable of being prepared in such a manner as to trap pressurized gas in cells include, for example, water soluble compounds such as salts, alkali halides, and alkaline earth metal halides. Specific salts useful in the present disclosure include, for example, sodium chloride, potassium chloride, potassium bromide, lithium chloride, cesium chloride, and the like. Typically, the cells containing the pressurized gas have a diameter of from about 5 micrometers to about 100 micrometers.

The substantially non-reactive gas can be infused into the cells of the soluble solid material to produce an effervescent agent useful in the present disclosure by first heating the starting material, such as a sugar, in a small amount of water until the material is dissolved. After dissolution of the material, the water is evaporated off leaving the material in a molten state. The molten material is then gasified by introducing a suitable gas, such as carbon dioxide, at a superatmospheric pressure into a sealed vessel containing the molten material. The molten material is agitated during gasification to ensure intimate contact between the molten material and the gas. Pressures of, for example, between about 50 psig (340 kPa) and about 1000 psig (6890 kPa) can be utilized to infuse the gas into the molten material. After gas infusion, the molten material is allowed to solidify while maintained in the sealed vessel to produce an effervescent agent. A suitable procedure of producing a gas-containing solid material is fully set forth in U.S. Pat. No. 4,289,794, the contents of which are incorporated herein by reference to the extent they are consistent (i.e., not in conflict) herewith. The above procedure can produce solid effervescent agents containing cells of pressurized gas from about 50 psig (340 kPa) to about 900 psig (6200 kPa) that, when exposed to urine or other body exudates, allow the release of the trapped gas. This trapped gas, when released, can interact with the surfactant material described herein. The fizzing element 150 can suitably include from about 0.1 grams to about 15 grams of effervescent solid material containing a pressurized gas.

In various aspects of the present disclosure, one or more of the substances described herein can be combined in an air laid material or in a coform material and incorporated into the absorbent article 20. As a specific example, tartaric acid can be combined with a coform on one layer with calcium carbonate on that or another layer. This material will then bubble vigorously when subjected to an aqueous solution. That bubbling is detectable to the wearer of the absorbent article 20 and signals that the absorbent article 20 has been insulted.

Physical sensation elements of this type are described in more detail in U.S. Pat. No. 7,002,055 to Long et al., the contents of which are incorporated herein by reference to the extent that they are consistent (i.e., not in conflict) herewith. The fizzing element 150 includes a surfactant and a system that, upon wetting with urine or other body exudates, produces a gas, such as carbon dioxide. The gas produced upon wetting with urine or other body exudates interacts with the surfactant to produce foam.

As noted above, the fizzing element 150 additionally includes a surfactant. The surfactant component is present as a foaming agent. When a gas, such as carbon dioxide, is produced from the gas generating system located in the fizzing element 150, the gas interacts with the surfactant to produce bubble-filled foam. This bubble-filled foam pushes against the skin of the wearer to alert the wearer to the insult of the absorbent article 20.

The surfactant used is not critical so long as it does not substantially irritate the skin upon contact. A wide variety of surfactants can be suitable for use in accordance with the present disclosure. For example, suitable surfactants include anionic surfactants, nonionic surfactants, amphoteric surfactants, cationic surfactants, and combinations thereof. Examples of suitable anionic surfactants include alkyl benzene sulfonates, alkyl sulfates, alkyl ether sulfates, sulfosuccinates, and combinations thereof. Examples of suitable nonionic surfactants include ethoxylated alcohols, fatty acid alkanolamides, ethoxylated alkanolamides, amine oxides, and combinations thereof. Examples of suitable amphoteric surfactants include alkyl betaines, amidobetaines, and combinations thereof. Examples of suitable cationic surfactants include alkylammonium halides. Generally, the fizzing element 150 will include from about 0.1 grams to about 15 grams of surfactant.

In one aspect of the present disclosure, the components included in the system capable of generating a gas, such as carbon dioxide, upon being wetted and/or the surfactant present in the fizzing element 150 can be encapsulated in a urine-or-other-body-exudates-soluble shell material prior to introduction into the fizzing element 150. For example, if the system capable of generating a gas upon being wetted includes an acid and a base, the acid and the base can be separately encapsulated in a soluble encapsulation material to keep the components separated until wetted. Alternatively, the acid and base components can be encapsulated together if reactivity between the acid and the base in the absence of a liquid is not a concern. The surfactant can be separately encapsulated, or can be encapsulated with the acid and/or the base. Additionally, encapsulation can be used with gas-impregnated effervescent agents alone or in combination with the surfactant.

The shell material used for encapsulation can be suitably constructed of a material such that it will release the encapsulated material (i.e., the acid, base, effervescent agent and/or surfactant) upon contact with urine or other body exudates. The urine or other body exudates can cause the shell material to solubilize, disperse, swell, or disintegrate, or the shell material can be permeable such that it disintegrates or discharges the encapsulated material upon contact with urine or other body exudates. Suitable shell materials include cellulose-based polymeric materials (e.g., ethyl cellulose), carbohydrate-based materials (e.g., starches and sugars) and materials derived therefrom (e.g., dextrins and cyclodextrins) as well as other materials compatible with human tissues.

The shell thickness can vary depending upon the material encapsulated, and is generally manufactured to allow the encapsulated component to be covered by a thin layer of encapsulation material, which can be a monolayer or thicker laminate, or can be a composite layer. The layer should be thick enough to resist cracking or breaking of the shell during handling or shipping of the product or during wear that would result in breakage of the encapsulating material. The material should also be constructed such that humidity from atmospheric conditions during storage, shipment, or wear will not cause a breakdown of the microencapsulation layer.

These and other modifications and variations to the present disclosure can be practiced by those of ordinary skill in the art, without departing from the spirit and scope of the present disclosure, which is more particularly set forth in the appended claims. In addition, it should be understood that aspects of the various aspects can be interchanged both in whole or in part. Furthermore, those of ordinary skill in the art will appreciate that the foregoing description is by way of example only, and is not intended to limit the disclosure so further described in such appended claims.

What is claimed:

1. A pant-like absorbent article, comprising:
an absorbent chassis defining a waist opening and first and second leg openings, the absorbent chassis including an absorbent assembly;
the absorbent chassis having a longitudinal length and the waist opening having an unstretched circumference, such that the longitudinal length is proportional to the unstretched circumference of the waist opening by a ratio less than 0.82; and
a wetness indicator for alerting a wearer to a release of liquid body exudates, the wetness indicator including a physical sensation agent responsive to liquid body exudates received by the absorbent article to facilitate a physical sensation against the wearer's skin for alerting the wearer to the wearer's release of liquid body exudates.

2. The absorbent article of claim 1, wherein the physical sensation is a temperature change.

3. The absorbent article of claim 1, wherein the physical sensation is fizzing.

4. The absorbent article of claim 1, wherein the wetness indicator is in overlaid relationship with the absorbent assembly.

5. The absorbent article of claim 1, further comprising a liner adapted for generally contiguous relationship with the wearer's skin during wearing of the absorbent article, and an outer cover, the wetness indicator being disposed between the liner and the outer cover.

6. The absorbent article of claim 1, wherein the absorbent article is configured for wear about a wearer's waist, the absorbent article having a front region, a back region and a crotch region interconnecting the front and back regions and extending generally longitudinally therebetween, the wetness indicator being longitudinally positioned generally within the crotch region of the absorbent article.

7. The absorbent article of claim 1, the wetness indicator further comprising a temperature element, wherein the physical sensation agent is a temperature change agent in the form of particles disposed on or within the temperature element of the wetness indicator.

8. The absorbent article of claim 7, wherein the temperature change agent comprises an endothermic material.

9. The absorbent article of claim 1, wherein the longitudinal length is proportional to the unstretched circumference of the waist opening by a ratio less than 0.80.

10. The absorbent article of claim 1, wherein the longitudinal length is proportional to the unstretched circumference of the waist opening by a ratio less than 0.78.

11. The absorbent article of claim 1, wherein the wetness indicator further includes a liquid absorbent body therein, the liquid absorbent body adapted to absorb liquid body exudates in the presence thereof so that the wetness indicator increases in thickness as liquid body exudates are absorbed, the wetness indicator having a first thickness when dry and a second thickness greater than the first thickness upon absorption of liquid body exudates.

12. The absorbent article of claim 11, wherein the second thickness is at least about three times greater than the first thickness.

13. The absorbent article of claim 11, wherein the second thickness is between about three and about twenty times greater than the first thickness.

14. The absorbent article of claim 11, wherein the second thickness is between about five and about fifteen times greater than the first thickness.

15. The absorbent article of claim 1, wherein the unstretched circumference of the waist opening is between about 450 and about 750 millimeters.

16. The absorbent article of claim 1, wherein the unstretched circumference of the waist opening is between about 500 and about 700 millimeters.

17. An article for personal wear, the article being capable of alerting a wearer to the wearer's release of liquid body exudates, the article comprising:
an outer cover, an absorbent assembly, a waist opening, and first and second leg openings, the article having a longitudinal length and the waist opening having an unstretched circumference;
a liner adapted for contiguous relationship with the wearer's skin due to the longitudinal length being proportional to the unstretched circumference of the waist opening by a ratio less than 0.82; and
a wetness indicator disposed between the liner and the outer cover, the wetness indicator including
an absorbent body disposed for absorbing liquid body exudates, whereby the wetness indicator swells as the absorbent body absorbs liquid body exudates, and
a physical sensation element including a physical sensation agent responsive to the liquid body exudates to facilitate a physical sensation against the wearer's skin, the physical sensation agent being disposed at least one of on or within the physical sensation element such that liquid body exudates absorbed by the physical sensation element of the wetness indicator are subjected to a physical sensation by the physical sensation agent at least one of prior to and upon absorption of liquid body exudates by the absorbent body.

18. The article of claim 17, wherein the physical sensation is a temperature change.

19. The article of claim 17, wherein the physical sensation is fizzing.

20. The article of claim 17, wherein the longitudinal length is proportional to the unstretched circumference of the waist opening by a ratio less than 0.80.

21. The article of claim 17, wherein the longitudinal length is proportional to the unstretched circumference of the waist opening by a ratio less than 0.78.

22. A method for producing an article for personal wear, the article being capable of alerting a wearer to the wearer's release of liquid body exudates, the method comprising:
producing a first disposable absorbent article of size n including no wetness indicator and having a size n longitudinal length and a waist opening with a size n unstretched circumference;
producing a second disposable absorbent article of size n-1 substantially equivalent in design to the first disposable absorbent article, the second disposable absorbent article including no wetness indicator and having a size n-1 longitudinal length and a waist opening with a size n-1 unstretched circumference; and
producing a third disposable absorbent article of size n including a wetness indicator and having a longitudinal length of the size n-1 longitudinal length ±5% and a waist opening unstretched circumference of the size n unstretched circumference ±5%.

23. The method of claim 22, the third disposable absorbent article further comprising a physical sensation agent.

24. The method of claim 22, the third disposable absorbent article further comprising a swellable wetness indicator.
